(19) United States
(12) Patent Application Publication
(10) Pub. No.: US 2020/0146712 A1
(43) Pub. Date: May 14, 2020

(54) DEVICE FOR GUIDING MEDICAL AND COSMETIC INJECTIONS

(71) Applicant: Nikola Ilja Milovancev, Novi Sad (RS)

(72) Inventor: Nikola Ilja Milovancev, Novi Sad (RS)

(21) Appl. No.: 16/190,078

(22) Filed: Nov. 13, 2018

(51) Int. Cl. A61B 17/34; A61M 5/42

(52) U.S. Cl. CPC A61B 17/3403; A61M 2210/0606; A61B 2017/3411; A61M 5/427

(57) ABSTRACT

The disclosure is directed a device and related methods for guiding medical injections. The device generally includes a disc portion and a handle portion extending therefrom. The disc extends from an aperture to a periphery, wherein dimensions of the aperture and periphery are selected to be useful for medical and cosmetic injections, such as administration of botulinum toxin and other applications. The device is used to identify and mark injection sites thereby planning the procedure and guiding the physician during administration of the injections.

### BACKGROUND

### Field of the Invention

The invention relates to medical devices; and more particularly, to a device for planning and guiding medical and cosmetic injections, such as, for example but not limitation, administration of botulinum toxin for medical and cosmetic applications.

### Description of the Related Art

The procedure for injecting botulinum toxin is both an art and science that only a qualified healthcare professional should perform.

There are forty-three muscles in one's face, and it's vital that the person who performs botulinum toxin injections understands and pinpoints the correct spots to optimize the treatment. Muscles which are commonly identified for receiving botulinum toxin injections include: procerus, corrugator, frontalis, and orbicularis oculi muscles.

For most procedures, a very thin needle is used to inject small amounts of botulinum toxin into specific muscles. By carefully choosing specific muscles, only the wrinkle-producing muscles are affected, preserving the patient's natural facial expressions.

Currently, physicians are approximating injection sites, for example, by asking a patient to contract the relevant muscles, and using a marking implement to apply markings on the face of a patient where a physician estimates the injection to be beneficial. However, using such conventional techniques often produces asymmetrical results, and other side effects which are generally problematic. There is no standardized protocol for determining injection sites for botulinum toxin injections.

There is a need for improved devices and related protocols that will improve consistency and repeatability, as well as symmetry of injection sites for use with the administration of botulinum toxin.

### SUMMARY

The disclosure is directed a device and related methods for guiding medical injections, and more particularly, for marking injection sites for subsequent injections. The device generally includes a disc portion and a handle portion extending therefrom. The dimensions of the aperture and periphery are selected to be useful for medical and cosmetic injections, such as administration of botulinum toxin. The device is used to identify and mark injection sites thereby planning the procedure and guiding the physician during administration of the injections.

In an illustrated embodiment, the device comprises: a disc portion and a handle portion; the disc portion extends radially from an aperture to a periphery, wherein the aperture is disposed at a center of the disc portion; the handle portion is coupled to and extends outwardly from the periphery of the disc portion; wherein the handle portion is bent relative to the disc portion at a bending line.

Other embodiments are further described herein.

### BRIEF DESCRIPTION OF THE DRAWINGS

The features and benefits of the invention will be further appreciated by one with skill in the art upon a thorough review of the illustrated and other embodiments as set forth in the appended detailed descriptions and drawings, wherein:

FIG. 1 shows a perspective view of a device for guiding medical and cosmetic injections in accordance with an illustrated embodiment;

FIG. 2 shows a side view of the device in accordance with the illustrated embodiment;

FIG. 3 shows a front view of the device in accordance with the illustrated embodiment;

FIG. 4 shows a top view of the device in accordance with the illustrated embodiment;

FIG. 5 shows a patient with injection site markings applied using the device in accordance with the illustrated embodiment of FIGS.1-4; and

FIG. 6 illustrates a preferred method for using the device.

### DETAILED DESCRIPTION

For purposes of explanation and not limitation, details and descriptions of certain preferred embodiments are hereinafter provided such that one having ordinary skill in the art may be enabled to make and use the invention. These details and descriptions are representative only of certain preferred embodiments, however, a myriad of other embodiments which will not be expressly described will be readily understood by one having skill in the art upon a thorough review hereof. Accordingly, any reviewer of the instant disclosure should interpret the scope of the invention by the claims, and such scope is not intended to be limited by the embodiments described and illustrated herein.

### General Embodiment

In a general embodiment, a device for guiding and planning medical and cosmetic injections is disclosed. The device is particularly useful for administration of botulinum toxin about the face of a patient, particularly the forehead area, for cosmetic applications, such as treating wrinkles, but also for medical applications such as, inter alfa, to treat excessive sweating or migraine headaches.

The device for guiding medical and cosmetic injections generally comprises: a disc portion and a handle portion; the disc portion extends radially from an aperture to a periphery, wherein the aperture is disposed at a center of the disc portion; the handle portion is coupled to and extends outwardly from the periphery of the disc portion; wherein the handle portion is bent relative to the disc portion at a bending line.

In an embodiment, the disc portion comprises a disc-diameter, wherein the disc-diameter is between 10.0 mm and 60.0 mm. In a preferred embodiment, the disc-diameter is 30.0 mm.

In another embodiment, the aperture comprises an aperture-diameter, wherein the aperture-diameter is between 2.0 mm and 10.0 mm. More preferably the aperture-diameter is 6.0 mm.

In another embodiment, the handle portion is bent relative to the disc portion at an angle between 45 and 179 degrees. More preferably, the handle portion is bent relative to the disc portion at an angle that is 154 degrees therebetween.

In other embodiments, the device may comprise no bend angle. In this regard, the handle portion can be provided configured within a common plane with the disc portion.

The device may comprise a thickness between 0.5 mm and 5.0 mm. Preferably, the thickness of the device is about 2.0 mm.

The device can be molded, or may be cast or computer numerical control (“CNC”)-cut from a sheet of material, and subsequently bent using heat, pressure, or a combination thereof. In this regard, the disc portion and the handle portion may consist of a single monolithic piece.

While the device may be fabricated from metal, such as aluminum, in a preferred embodiment the device is fabricated from a plastic, such as acrylic or other polymeric compositions. In a preferred embodiment, the device is fabricated form a polycarbonate material, for example, MACROLON®.

In the preferred embodiment, the device is fabricated from a transparent material to aid in placement of the device relative to features of a patient's anatomy or skin folds, or relative to markings created by the physician.

### Illustrated Embodiment

Now turning to the drawings, FIG. 1 shows a perspective view of a device for guiding medical and cosmetic injections in accordance with the illustrated embodiment. The device comprises a disc portion 10 and a handle portion 20. The disc portion extends radially from an aperture 11 to a periphery 13. The handle portion is coupled to the disc portion at the periphery of the disc portion and extends outwardly therefrom.

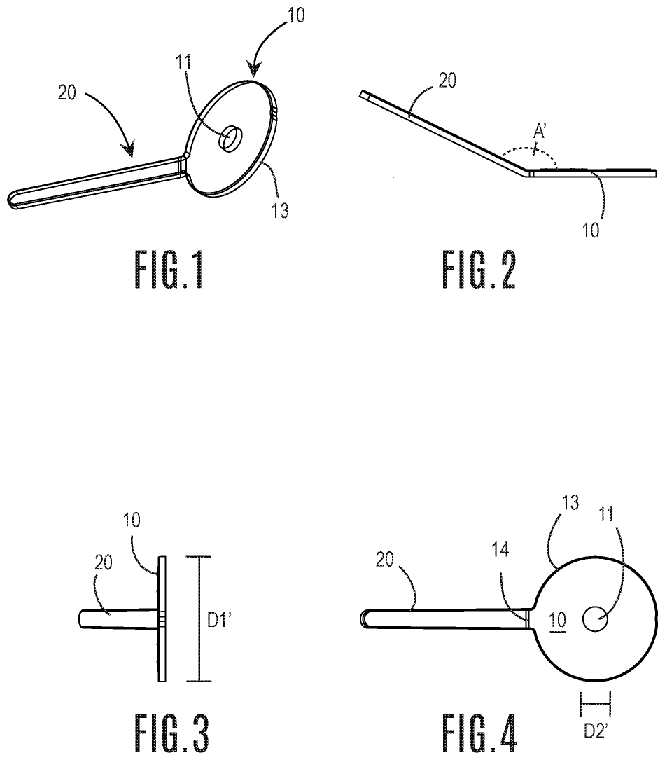

FIG. 2 shows a side view of the device in accordance with the illustrated embodiment. The handle portion 20 is shown as being coupled to, and extending from, a periphery of the disc portion 10. The handle portion is bent relative to the disc portion, wherein the bend forms an angle therebetween, the angle is hereinafter referred to as the “bend-angle (A′)”. As disclosed above, the bend-angle A′ may comprise an angle between 45 and 179 degrees, preferably 154 degrees.

FIG. 3 shows a front view of the device in accordance with the illustrated embodiment. The disc portion 10 comprises a disc-diameter (D1′). The handle 20 is shown extending from the disc portion. As disclosed above, the disc-diameter D1′ may comprise between 10.0 mm and 60.0 mm, preferably 30.0 mm. The diameter is associated with an area of tissue spanning the injection site for which a certain volume and/or number of international units of botulinum toxin solution is effective. It is proposed that the disc portion should comprise a disc-diameter of about 30.0 mm; though the diameter may be adjusted for specific botulinum toxin solutions and dosing variants.

FIG. 4 shows a top view of the device in accordance with the illustrated embodiment. Here, the disc portion 10, aperture 11, and periphery 13 may be further appreciated from the top view. The handle portion 20 is shown extending outwardly from the disc portion 10 at or near the bending line 14. The bending line can be created by heating a device cut from a planar sheet and subsequently bending at the bending line using a brake or other mechanical instrument known to one with skill in the art. Also illustrated in FIG. 4 is aperture diameter D2′. The aperture diameter should be dimensioned for enabling a suitable marking using a conventional marking element. As disclosed above, the aperture diameter D2′ may comprise between 2.0 mm and 10.0 mm, preferably 6.0 mm.

### Methods for Using the Device

In accordance with another aspect, a method is disclosed for using the device in conjunction with a medical or cosmetic procedure. Here, the procedure comprises administration of a botulinum toxin solution, such as those known by one with skill in the art and commercially available, and the application may be cosmetic or medical, such as treating wrinkles, or treating excessive sweating at the forehead region of a patient, respectively.

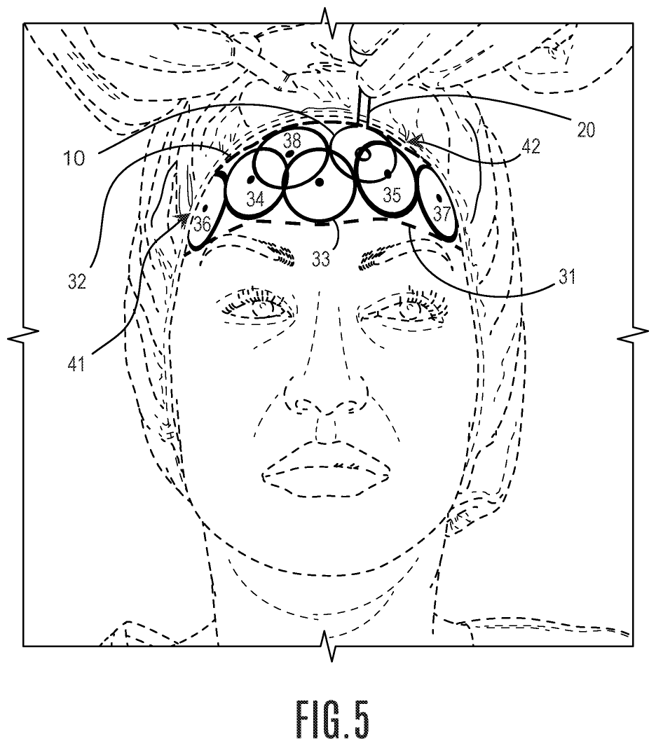

FIG. 5 shows a patient with various markings created using the device.

### EXAMPLE 1

### Forehead Lines

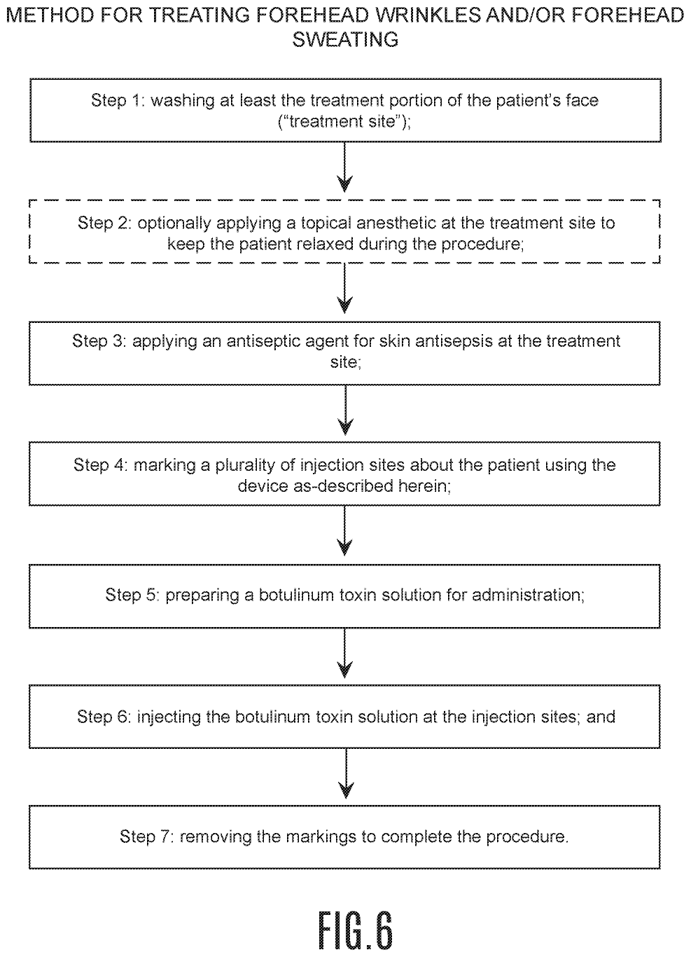

As illustrated in FIG. 6, a method for using the device in accordance with a procedure for treating forehead wrinkles and/or forehead sweating comprises:

Step 1: washing at least the treatment portion of the patient's face (“treatment site”);

Step 2: optionally applying a topical anesthetic at the treatment site to keep the patient relaxed during the procedure;

Step 3: applying an antiseptic agent for skin antisepsis at the treatment site;

Step 4: marking a plurality of injection sites about the patient using the device as-described herein;

Step 5: preparing a botulinum toxin solution for administration;

Step 6: injecting the botulinum toxin solution at the injection sites; and

Step 7: removing the markings to complete the procedure.

During Step 1, the physician or an aid will generally apply soap and water to clean any makeup or other elements from the treatment site. Alternatively, or additionally, alcohol may be used to dissolve any foreign matter and to clean the area in preparation for treatment.

Topical anesthetics known to one having skill in the art may be optionally applied. Because the needle(s) used in botulinum toxin injections are very small, for example 30 gauge to 32 gauge, there is often no need for topical anesthetic. However, for patients experiencing a first treatment, or those weary of needles, a topical anesthetic such as, for example and not limitation, prilocaine, lidocaine, or even ice, can be applied. It is preferred that the patient is calm and relaxed during the procedure in order to avoid tension in the treatment area muscles which could introduce errors in the treatment results. As such, a topical anesthetic is preferably applied to numb the treatment area.

An antiseptic agent, such as isopropyl alcohol, is generally applied to reduce the incidence of surgical site infections (S SI) at the treatment site.

With Steps 1 thru 3 complete, the next task is to prepare the patient with markings for guiding injections during the procedure. In this regard, the device as described herein is utilized to plan and mark the required injection sites about the patient's frontalis muscle (forehead region).

The physician begins by asking the patient to maximally elevate the eyebrows. With the patient's eyebrows maximally elevated, the physician then marks the inferior border and superior border of the frontalis muscle by marking the lowest and highest frontal wrinkles, respectively, using a marking element, thereby creating an inferior frontalis marking (31, FIG. 5) and a superior frontalis marking (32, FIG. 5) which will collectively define the upper and lower boundaries of the treatment site.

Next, with reference to FIG. 5, the physician asks the patient to relax the forehead as the device is intended to be used with the forehead in a relaxed state. The device is used to symmetrically plan a plurality of injection sites within the area of the treatment site. In this regard, the device is held by the physician at the handle portion (20, FIG. 1) thereof, and the disc portion (10, FIG. 1) is applied to the forehead of the patient. The physician applies the device such that the disc is placed at the center and lowest position within the treatment site (“first position”); i.e. the disc portion of the device is centered within the treatment site and along the inferior frontalis marking 31 to establish a first trace 33 of the device at the first position using the marking element. Each of the aperture and the periphery of the device are marked with the device placed at the first position. The aperture provides a dot marking to identify an injection site, whereas the periphery is traced to provide an annular marking for identifying the radial effective boundary of the respective injection site.

The device is then moved horizontally (left and right) to place the periphery of the device at a second and subsequent positions 34; 35; 36; 37 as shown, each of which is located along the inferior frontalis marking 31, and adjacent to the peripheral trace of the previous marking or the first trace; whereby at each position along the inferior frontalis marking a subsequent marking is made at the aperture and the periphery of the device.

Markings (tracings of the device periphery and aperture) are applied along the left-and right-sides of the first trace 33 until the frontalis muscle is completely marked with a plurality of device-markings extending along a “lower disc-lane 41” as shown in FIG. 5. For most patients, the method requires five disc-markings in the lower disc-lane.

Again referencing FIG. 5, where there are portions of the treatment site that are between the superior frontalis marking 32 and the plurality of horizontal device-markings (33; 34; 35; 36; 37) within the lower disc-lane, the device is placed with the periphery thereof overlapping with two adjacent device-markings and aligning with the superior frontalis marking 32, and an upper marking 38 is created by marking the aperture and tracing the periphery of the device. Note that overlapping of device-markings along the horizontal lanes is not helpful, however, overlapping the markings of the upper- and lower-disc lanes helps to cover the entire treatment site and can strengthen the procedure for delivering longer lasting results. This is repeated and upper markings are created as necessary to cover the entire treatment site and define the “upper disc-lane 42” as shown in FIG. 5. For most patients, two to four markings will be required in the upper disc-lane.

Now, with markings created, the physician may prepare a botulinum toxin solution. Alternatively, the composition is prepared prior to the patient's visit. Preparing the botulinum toxin solution may require dilution and loading a syringe with the composition. As mentioned above, a 30-gauge or 32-gauge needle is preferred (though 27-34 gauge can be used).

The needle is placed at each of the injection sites identified by the aperture-dot markings previously applied. The physician will inject a botulinum toxin solution at each injection site.

While various details, features, and combinations are described in the illustrated embodiments, one having skill in the art will appreciate a myriad of possible alternative combinations and arrangements of the features disclosed herein. As such, the descriptions are intended to be enabling only, and non-limiting. Instead, the spirit and scope of the invention is set forth in the appended claims.

### FEATURE LIST

disc portion (10)
aperture (11)
periphery (13)
bending line (14)
handle portion (20)
inferior frontalis marking (31)
superior frontalis marking (32)
first trace (33)
markings at second and subsequent positions (34; 35; 36; 37)
(38)
lower disc-lane (41)
upper disc-lane (42)
bend-angle (A′)
disc-diameter (D1′)
aperture-diameter (D2′)

What is claimed is:

1. A device for guiding medical and cosmetic injections, comprising:
a disc portion (10) and a handle portion (20);
the disc portion extending radially from an aperture (11) to a periphery (13), wherein the aperture is disposed at a center of the disc portion;
the handle portion coupled to and extending outwardly from the periphery of the disc portion;
wherein the handle portion is bent relative to the disc portion at a bending line (14).

2. The device of claim 1, the disc portion comprising a disc-diameter (D1′), wherein the disc-diameter is between 10.0 mm and 60.0 mm.

3. The device of claim 2, wherein the disc-diameter is 30.0 mm.

4. The device of claim 1, the aperture comprising an aperture-diameter (D2′), wherein the aperture-diameter is between 2.0 mm and 10.0 mm.

5. The device of claim 4, wherein the aperture-diameter is 6.0 mm.

6. The device of claim 1, wherein the handle portion is bent relative to the disc portion forming a bend-angle therebetween, the bend-angle comprising between 45 and 179 degrees.

7. The device of claim 6, wherein the bend-angle is 154 degrees.

8. The device of claim 1, comprising a thickness between 0.5 mm and 5.0 mm.

9. The device of claim 8, wherein the thickness is 2.0 mm.

10. The device of claim 1, wherein the disc portion and the handle portion comprise a monolithic piece.

11. A method for treating forehead wrinkles and/or excessive sweating of a forehead of a patient, the method comprising:
Step 1: washing at least a portion of the patient's face at a treatment site;
Step 2: optionally applying a topical anesthetic at the treatment site to keep the patient relaxed during the procedure;
Step 3: applying an antiseptic agent for skin antisepsis at the treatment site;
Step 4: marking a plurality of injection sites about the patient with a device comprising: a disc portion and a handle portion, the disc portion extending radially from an aperture to a periphery, wherein the aperture is disposed at a center of the disc portion, the handle portion coupled to and extending outwardly from the periphery of the disc portion, wherein the handle portion is bent relative to the disc portion at a bending line;
Step 5: preparing a botulinum toxin solution for administration;
Step 6: injecting the botulinum toxin solution at the injection sites; and
Step 7: removing the markings.
